(19) United States
(12) Patent Application Publication
(10) Pub. No.: US 2005/0182370 A1
(43) Pub. Date: Aug. 18, 2005

(54) Prefilled syringe with plunger backward movement limiting mechanism

(76) Inventor: Seiichi Hato, Kanagawa (JP)

Correspondence Address: DAVID W. HIGHET VP & CHIEF IP COUNSEL;BECTION DICKINSON AND COMPANY, 1 BECTON DRIVE MC 110, FRANKLIN LAKES, NJ, 07417-1880, US

(21) Appl. No.: 10/507,767

(22) PCT Filed: Mar. 11, 2003
(86) PCT No.: PCT/JP03/02851

(30) Foreign Application Priority Data
Mar. 15, 2002 (JP) 2002-72083

(51) Int. Cl. A61M 5/178

(52) U.S. Cl. 604/213

(57) ABSTRACT

A risk of injection liquid contamination due to contact of injection liquid in an injection-liquid filled area in a barrel with the surface in an injection-liquid unfilled area in the barrel by causing a plunger of a prefilled syringe to be inadvertently withdrawn by mishandling which may leads to medical malpractice, is eliminated by the invention. Withdrawal of a plunger 14 is limited by engagement of a barrel 12 in which a withdrawal-limiting member 48 is provided and a the plunger 14 in which a withdrawal-limiting member 54 is provided. As a result of this, contact of the injection liquid L prefilled to a liquid-filled area 32 in the barrel 12 with the surface of a liquid-unfilled area 34 in the barrel by withdrawal of the plunger 14 is prevented.

### TECHNICAL FIELD

The present invention relates to a syringe in which injection liquid is prefilled (prefilled syringe), and more particularly, the prefilled syringe which is provided with a means for preventing contamination of the prefilled injection liquid.

### BACKGROUND TECHNIQUE

For a conventional type of prefilled syringe, injection liquid is filled into the syringe barrel and a sliding plug (of the liquid-injecting plunger) is inserted in the barrel, usually under a sterilized condition, and thereafter, a plunger rod is fixed to the sliding plug and works such as labeling and packaging are done for shipment under a non-sterilized condition.

A syringe in which some specific injection liquid is prefilled may be finally sterilized by heating using an autoclave etc. after packaging. However, in other type of syringe in which injection liquid unable to be sterilized by heating is prefilled, bacteria or other microorganisms are considered to have started to adhere to the syringe immediately after it was brought out from the sterilized condition. Namely, in such condition, only the liquid-filled space sealed by the sliding plug (of the plunger) in the barrel is kept sterilized, and there is a possibility that microorganisms adhere to the backward area of the sliding plug, even if the backward area is inside the barrel, before the prefilled syringe is used.

Consequently, in case of the above-mentioned syringe, when the sliding plug is withdrawn backward, there is a risk that bacteria or other microorganisms adhering to the inner surface of the barrel may contaminate injection liquid and the syringe is therefore hazardous. The control of such risk has been depending on the careful handling of the prefilled syringe by care providers such as physicians and nurses, but their mishandling has been reasonably anticipated. However, in reality, injection liquid was contaminated due to the mishandling, that may result in medical malpractice. Even if such contamination of injection liquid and medical malpractice due to it occur, it was difficult to determine that such malpractice was attributable to the mishandling of the prefilled syringe and the problem has therefore not been clearly recognized.

The invention is intended to clarify where the problem exists and to provide a prefilled syringe entirely free from a risk of injection liquid contamination due to mishandling and resultant medical malpractice.

### DISCLOSURE OF THE INVENTION

The invention provides the prefilled syringe comprising a barrel having a closed front-end wall with a small passageway through which injection liquid is discharged and a rear opening, a plunger including a sliding plug liquid-tightly contacting with the inner surface of the barrel and partitioning the inside of the barrel into a liquid-filled area which is formed between the plunger and the closed front-end wall and in which injection liquid is filled and sealed and a liquid-unfilled area in the back of the liquid-filled area and a plunger rod extending backward from the sliding plug through the rear opening of the barrel, and a plunger withdrawal-limiting member fixed to the barrel, which limits withdrawal of the plunger by engaging the plunger and prevents injection liquid from contacting with the surface of the liquid-unfilled area in the barrel.

Referring more particularly, the plunger withdrawal-limiting member which is provided at the rear opening of the barrel and extends from the rear opening to the inside of the barrel for a certain length, can contact with the backward-moving sliding plug of the plunger so that withdrawal of the plunger is limited.

It is also possible that the plunger withdrawal-limiting member provided at the rear opening of the barrel is in the form of an annular withdrawal-limiting flange comprising an opening for the plunger rod which extends inwardly in the radial direction from the periphery of the rear opening of the barrel and is sized to allow the plunger rod to pass therethrough, while a stopper member extending outwardly in the radial direction from the plunger rod is provided. As a result of this, withdrawal of the plunger is limited by engagement of the stopper member with the withdrawal-limiting flange.

In the above case, it is possible to stably push and handle the plunger by allowing at least a portion of the outer peripheral surface of the withdrawal-limiting member to closely contact with the inner peripheral surface of the barrel. It is also possible that the position in which the withdrawal-limiting member is fixed to the plunger rod is adjusted at needs in accordance with the quantity of injection liquid to be prefilled.

In other form of embodiment, the withdrawal-limiting member may be made in the form of a cylindrical member in which a male screw is provided on the outer peripheral surface thereof, and on the other hand, the plunger withdrawal-limiting member may be formed in the form of an annular flange in which the inner peripheral surface of the flange defines an opening having a size corresponding to the plunger withdrawal-limiting member, the annular flange being provided at the rear end opening of the barrel and extending inwardly in a radial direction from the peripheral edge of the opening and a female screw to be mated with the male screw is formed on the inner peripheral surface of the flange. In this form of syringe, when the plunger is withdrawn for a predetermined distance over a predetermined position, the stopper member engages the withdrawal-limiting member, thus preventing withdrawal of the plunger. If the male screw of the stopper member is further turned and fitted into the female screw on the plunger withdrawal-limiting member the plunger can be further withdrawn.

Additionally, it is possible that the stopper member is formed in the form of a leaf spring shaped member which extends diagonally backward from a predetermined location in the plunger rod so that when the plunger rod is inserted into the barrel through the opening for the plunger rod the leaf spring is deflected by the surface of the opening for the plunger rod and after passing through the same opening the leaf spring returned to its original shape to act the function to limit plunger withdrawal.

Furthermore, it is possible that the stopper member is formed in a member projecting radially outward from the plunger rod and a notch through which the stopper member passes may be formed at the opening of the withdrawal-limiting flange so that the function of plunger withdrawal can be released.

### BRIEF EXPLANATION OF THE DRAWINGS

FIG. 1 is a sectional side elevational view of a prefilled syringe arrangement in accordance with the first embodiment of the invention;

FIG. 2 is a sectional side elevational view of a prefilled syringe arrangement in accordance with the second embodiment of the invention;

FIG. 3 is a sectional side elevational view of a prefilled syringe arrangement in accordance with the third embodiment of the invention;

FIG. 4 is a schematic illustration view of a prefilled syringe arrangement in accordance with the fourth embodiment of the invention;

FIG. 5 is a schematic illustration view of a prefilled syringe arrangement in accordance with the fifth embodiment of the invention; and

FIG. 6 is a schematic illustration view of the prefilled syringe according to the sixth embodiment of the invention in which the side view of the withdrawal-limiting flange is also shown.

### BEST MODE FOR CARRYING OUT THE INVENTION

The mode for carrying out the prefilled syringe according to the invention is explained below, referring to the attached drawings.

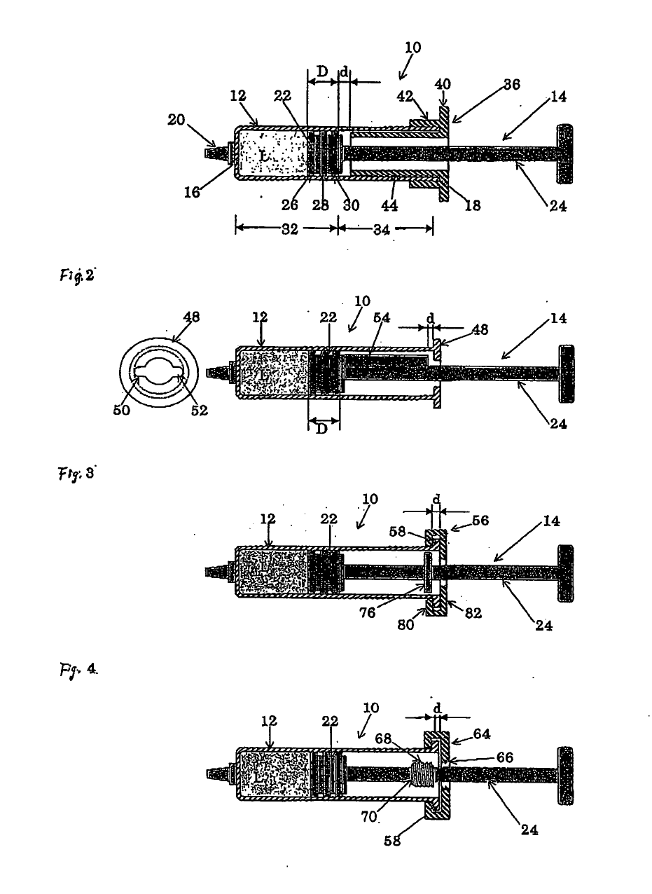

FIG. 1 shows the first mode for carrying out the prefilled syringe 10 according to the invention. As illustrated, the prefilled syringe 10 has a barrel 12 in a cylindrical form and the plunger 14 to be inserted into the barrel. The barrel has the front-end wall 16, the tip of which has a small passageway (not shown in the FIG. 1) through which the injection liquid is discharged, and the rear opening 18 through which the plunger 14 is received. A barrel tip cover 20 is mounted on the outer surface of the tip of the front-end wall 16. The plunger 14 has a sliding plug 22 at its tip which is able to slidably move along the inner surface of the barrel 12 and a plunger rod 24 which extends outwardly from the sliding plug 22 to the outside of the barrel through the rear opening 18 at the rear end of the barrel. FIG. 1 shows the completed prefilled syringe before use. In this state, the sliding plug 22 partitions the inside of the barrel 12 into a liquid-filled area 32 where the injection liquid L is filled and sealed and a liquid-unfilled area 34 in the back of the area 32. In the illustrative embodiment shown in the FIG. 1, the sliding plug 22 has a generally cylindrical form and three ring-shaped ribs 26, 28 and 30 are provided around the outer surface of the sliding plug with keeping predetermined spaces in the axial direction therebetween. As the ribs liquid-tightly contact with the inner surface of the barrel 12, so that the liquid-filled area 32 in which the injection liquid is filled and sealed is formed between the front-end wall 16 of the barrel and the sliding plug 16. Namely, in this state, the liquid-filled area 32 is formed between the front-end wall 16 and the right side rib 30 and the liquid-unfilled area 34 is formed between the right side rib 30 and the rear opening 18 of the barrel.

As illustrated in the FIG. 1, a withdrawal-limiting member 36 to limit withdrawal of the plunger 14 is provided around the rear opening 18 of the barrel. The withdrawal-limiting member has a base plate 40 in an annular form, which is abutted to the end edge of the rear opening 18, a outer cylindrical portion 42 which extends from the base plate toward the tip of the barrel along the outer surface of the barrel and an inner cylindrical portion 44 which extends longer than the outer cylindrical portion 42 from the base plate toward the tip of the barrel around the inner surface of the barrel. These outer and inner cylindrical portions are secured to the barrel by pinching the side wall of the barrel.

The front-end of the inner cylindrical portion 44 is spaced from the rear end of the sliding plug 22 for a distance of d. Therefore, the sliding plug 22 is able to move rearward by the distance d from the predetermined position shown in FIG. 1 and when the sliding plug is moved rearward for the distance d it abuts or engages the front end of the inner cylindrical portion 44 of the withdrawal-limiting member 36, thus further withdrawal being prevented. The distance d is determined to be shorter than a distance D between the front-end of the sliding plug (more accurately, the position where the left side rib 26 liquid-tightly contacts with the inner surface of the barrel) and the position where the rear end of the right side rib 30 liquid-tightly contacts with the inner surface of the barrel so that when the sliding plug is withdrawn no contact between injection liquid L and the inner surface in the liquid-unfilled area in the barrel.

For the above-mentioned prefilled syringe, the plunger 14 and the withdrawal-limiting member 36 may be formed from a general-purpose resin such as polypropylene and polyethylene using injection molding and the sliding plug 22 is preferably be made from rubber or similar materials.

FIG. 2 shows other mode for carrying out the prefilled syringe 10 according to the invention.

In this type of prefilled syringe, an annular withdrawal-limiting flange 48 is integrally molded with the cylindrical barrel 12 in the rear end therefore and an opening through which the plunger rod 24 passes is provided in the central portion of the withdrawal-limiting flange 48. In addition, a pair of notches 50 and 52 are formed diametrically in the diameter of the inner peripheral edge of the flange 48. On the other hand, on the surface of the plunger rod 24, a withdrawal-limiting member 54 extends backward from the rear-end of the sliding plug 22. The withdrawal-limiting member 54 has a shape and a size corresponding to that of the notches 50 and 52 so that the member 54 is able to pass through the notches. Therefore, after injection liquid is prefilled and the sliding plug 22 is loaded, the plunger rod 24 is inserted into the barrel with the withdrawal-limiting part 54 passing through the notch 50 or 52, and then the front-end of the plunger rod can be connected to the sliding plug 22. Between the rear-end of the withdrawal-limiting member 54 and the inner surface of the withdrawal-limiting flange 48, the space d keeping the same relation of D>d as shown in the embodiment in FIG. 1 is provided.

FIG. 3 shows the third mode for carrying out the prefilled syringe according to the invention.

In this type of prefilled syringe, different from the prefilled syringe shown in FIG. 2, the withdrawal-limiting flange 56 is separately formed from the barrel 12 and fixed to a flange 58 formed radially and outwardly of and integrally with the rear end of the barrel. It is possible that in the same manner as the flange holding members disclosed in the U.S. Pat. No. 2,814,983 and U.S. Pat. No. 2,845,426, etc., the withdrawal-limiting flange 56 may be an annular form comprising a front annular plate 80 and a rear annular plate 82 between which a space for holding the flange 58 of the barrel is provided. The withdrawal-limiting flange 56 is secured to the barrel in the following manner. Firstly, an aperture (not shown in FIG. 3) extending in radial direction is provided in a portion of the annular form. The withdrawal-limiting flange 56 is then positioned at right angle to the axis of the barrel 12 with the aperture being aligned with the flange 58 of the barrel, and the flange 56 is pushed onto the aperture and then the flange 58 of the barrel and the plunger rod 24 are passed through the aperture into the inside of the withdrawal-limiting flange 56 so that the flange 58 of the barrel is held in the space between the front plate 80 and the rear plate 82.

The withdrawal-limiting member 76 in a disc form is fixed to the plunger rod 24 at the distance of d from the rear-end annular plate 82 of the withdrawal-limiting flange 56. In this mode of the invention, by fixing the withdrawal-limiting member at a proper location in the axial direction of the plunger rod 24 depending on the quantity of the injection liquid to be filled in the prefilled syringe, it is possible to provide the withdrawal-limiting member 76 at the distance of d from the rear-end annular plate 82 of the withdrawal-limiting flange. The withdrawal-limiting member 76 in this mode according to the invention may be in a fan shape as well as in a disc shape, and in either case, it is possible to forward the plunger 14 into the barrel 12 in a stable manner by keeping the outer periphery of the withdrawal-limiting member close to the inner surface of the barrel.

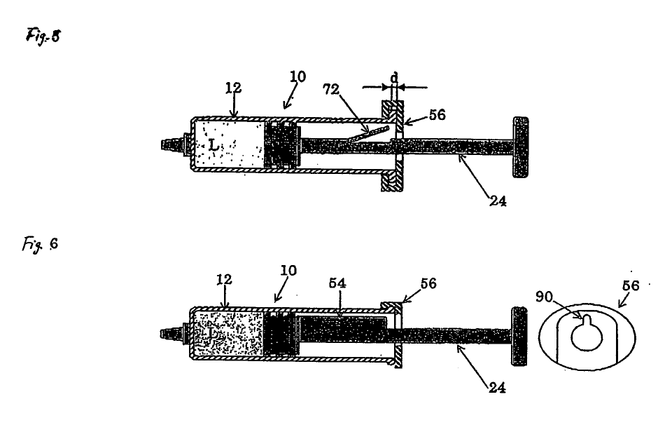

FIG. 4 shows the fourth mode for carrying out the prefilled syringe according to the invention.

In this type of prefilled syringe 10, a withdrawal-limiting flange 64 similar to the withdrawal-limiting flange 56 shown in FIG. 3 is mounted on the flange 58 which is integrally molded with the rear-end of the barrel 12 and radially extends. The difference of this type of prefilled syringe from that shown in FIG. 3 is that a female screw 66 is formed in the inner periphery of the withdrawal-limiting flange 64 and a male screw 68 fitting into the female screw 64 is correspondingly formed on a withdrawal-limiting member 70 which is fixed to the plunger rod 24.

Between the rear-end of the withdrawal-limiting member 70 and the front surface of the rear annular plate of the withdrawal-limiting flange 64, a space d is provided with keeping the same relation of D>d as shown in the mode in FIG. 1 and when the plunger 14 withdraws from a predetermined position by the distance d, the withdrawal-limiting member 70 abuts the withdrawal-limiting flange 64 to prevent further withdrawal. In order to further withdraw the plunger 14 and draw out the same from the barrel 12, the male screw of the withdrawal-limiting member 70 is turned and fitted into the female screw 66 of the withdrawal-limiting flange.

FIG. 5 shows the fifth mode for carrying out the prefilled syringe according to the invention. This type of prefilled syringe 10 is similar to that shown in FIG. 3, but a withdrawal-limiting member 72 fixed to the plunger rod 24 is in the form of leaf spring projecting diagonally backward. That is, when the plunger rod 24 is passed into to the inside of the barrel through the circular opening formed in the withdrawal-limiting flange 56, the leaf spring of the withdrawal limiting member 72 is deflected by the edge of the opening so that the leaf spring of the withdrawal-limiting member 72 passes through the opening into the barrel. After passage into the barrel, the leaf spring of the withdrawal-limiting member is sprung back to the form projecting diagonally backward, as illustrated in the FIG. 5. In this state, when the plunger withdraws from the predetermined position by the distance d, the rear-end of the leaf spring of the withdrawal-limiting member 72 abuts the withdrawal-limiting flange 56 to prevent further withdrawal.

The prefilled syringe shown in FIG. 6 is substantially identical to that shown in FIG. 2, but it is different from the syringe in FIG. 2 in the fact that only one notch 90 is formed in the opening for the plunger rod passage in the withdrawal-limiting flange 56 (shown as oval-shaped in FIG. 6). The withdrawal-limiting member 54 fixed to the plunger rod 24 can pass through the notch 90 so that the plunger rod 24 is moved forward into the inside of the barrel 12 and is withdrawn from the barrel.

### INDUSTRIAL APPLICABILTY

The prefilled syringe according to the invention has the above-mentioned constitution and through its simple structure surely prevents accidental withdrawal of the plunger due to mishandling. If the plunger is inadvertently withdrawn beyond the predetermined position, the injection liquid sealed in the liquid-filled area in the barrel may contact with the surface of the liquid-unfilled area (which bacteria etc. possibly adhere to), resulting in a risk of contamination. The invention can totally avoid such a risk and therefore prevent the occurrence of medical malpractice attributable to the contamination.

To prevent the contamination caused by bacteria, conventionally, the method involving heating sterilization using autoclave etc. has been employed after packing the produced prefilled syringe. However, it is impossible to apply the heating sterilization to some injection liquids and for the syringe prefilled with such liquids, the risk of contamination of such injection liquid is unavoidable. Therefor, the invention is especially beneficial to such prefilled syringe.

Also for the prefilled syringe in which an injection liquid suitable for heating sterilization is sealed, the heating sterilization process involves incremental facility investment and production cost. In this regard, the invention makes it possible to provide users with safer prefilled syringes not needing such costly process and is extremely beneficial to them.

1. A prefilled syringe comprising:
a barrel having a closed front-end wall with a small passageway through which injection liquid is discharged and a rear opening;
a plunger including a sliding plug liquid tightly contacting with the inner surface of the barrel and partitioning the inside of the barrel into a liquid-filled area which is formed between said plunger and said closed front-end wall and in which the injection liquid is filled and sealed, and a liquid-unfilled area in the back of the liquid-filled area and a plunger rod extending backward from said sliding plug through said rear opening of the barrel; and
a flange fixed to the rear opening of said barrel, a portion of which extends for a predetermined length into the barrel to limit plunger withdrawal so that the injection liquid is prevented from contacting with the surface of said liquid-unfilled area in the barrel by engaging said plunger.

2. (canceled)

3. The prefilled syringe according to claim 1, wherein said flange has an opening for said plunger rod extending inwardly in the radial direction from the periphery of said rear end opening of the barrel and being sized to allow said plunger rod to pass therethrough, and said plunger has a stopper member extending outwardly in the radial direction from the plunger rod, withdrawal of said plunger being limited by contact of said stopper member with said flange.

4. The prefilled syringe according to claim 3, wherein at least a portion of the outer peripheral surface of said stopper member is designed to make a close contact with the inner peripheral surface of said barrel.

5. The prefilled syringe according to claim 3, wherein said stopper member is in the form of a plate provided at right angles to the axis of the plunger rod and is able to be fixed to a required position in the axial direction of said plunger rod.

6. The prefilled syringe according to claim 3, wherein said stopper member is in the form of a cylindrical member having a male screw provided on the outer peripheral surface thereof, and a female screw mating with said male screw is formed at the opening for the plunger rod passage provided in said withdrawal-limiting flange.

7. The prefilled syringe according to claim 3, wherein said stopper member is a leaf spring extending diagonally backward from a predetermined position in said plunger rod, said leaf spring is adapted to be deflected by the surface of said opening for the plunger rod passage to be passed through the same opening when the plunger rod is inserted into said barrel through the same opening and is returned to an original shape after passing through the same opening.

8. The prefilled syringe according to claim 3, wherein said stopper member projects radially outward from said plunger rod and a notch for allowing said stopper member to pass through is formed at said opening for the plunger rod passage in said withdrawal-limiting flange.

9. The prefilled syringe according to claim 1, wherein the distance D between a portion on the outer peripheral surface of the sliding plug which is nearest to the closed front-end wall of said barrel and a portion on the same which is farthest from the closed front-end wall of the barrel and the distance d between the predetermined position of said plunger and the position where said plunger is withdrawn from said predetermined position and then engages said flange so that withdrawal of said plunger is prevented are adapted to keep the formula D>d.

10. The prefilled syringe according to claim 4, wherein said stopper member is in the form of a plate provided at right angles to the axis of the plunger rod and is able to be fixed to a required position in the axial direction of said plunger rod.
